(19) United States
(12) Patent Application Publication
(10) Pub. No.: US 2006/0213525 A1
(43) Pub. Date: Sep. 28, 2006

(54) Earplug and headgear with the earplug

(75) Inventor: Kimio Matsumoto, Osaka (JP)

Correspondence Address: KODA & ANDROLIA, 2029 CENTURY PARK EAST, SUITE 1140, LOS ANGELES, CA, 90067, US

(73) Assignee: Yamamoto Kogaku Co., Ltd.

(21) Appl. No.: 11/336,064

(22) Filed: Jan. 20, 2006

(30) Foreign Application Priority Data
Jan. 28, 2005 (JP) 2005-22082

(51) Int. Cl. A61F 11/06

(52) U.S. Cl. 128/867

(57) ABSTRACT

The present invention provides an earplug which can reduce resistance force of the water, rain and wind, and a time loss due to the resistance force in sports competing for speed and which is not dropped due to reduction in its holding power and which can reduce vibration sound generated when something touches or hits the earplug, prevent the vibration sound from being transferred to the ear hole of a user and protect a sense of hearing satisfactorily. Furthermore the present invention provides a headgear with the earplug in order to prevent the earplug from being dropped and lost or left behind. The earplug of the present invention is characterized in that the earplug body is connected to an arm 2 at an angle of 90±45°. The arm 2 to which the earplug body 1 is connected at the angle of 90±45° is connected to one end of a cord 3. Arms 2 and 2 to which earplug bodies 1 and 1 are connected at an angle of 90±45° are connected to both ends of cord 3. A headgear with the earplug of the present invention is connected to the cord 3 of the earplug.

### BACKGROUND OF THE INVENTION

1. Field of the Invention

The present invention relates to an earplug and a headgear with the earplug which is used to prevent the water, rain, wind and the like from entering into ear holes during sports activities and the like, or used to protect a sense of hearing in a noisy place.

2. Description of the Related Art

Conventionally, as this kind of earplug, there has been an ear plug in which a thin and soft umbrella-shaped wing panel 12 having a small diameter is provided at an end of a thin shaft 11 formed of a rubber or a flexible synthetic resin of vinyl chloride or polyethylene and having an appropriate length, and a wing panel 13 having a diameter larger than that of the wing panel 12 and a wing panel 14 having a diameter larger than that of the wing panel 13 are provided sequentially at appropriate intervals backward and integrally molded with the thin shaft 11 as shown in FIGS. 9 and 10 (for example, Japanese Utility Model Publication No. 38-19297 (P. 1, FIGS. 1 and 2)).

When the above earplug is inserted into the ear hole, since it can be inserted and stopped at the wing panel where a diameter of the ear hole fits, it can be appropriate for any ear hole regardless of the diameter size of the ear hole of a user.

Furthermore, as another example of this kind of earplug, there has been an ear plug in which insertion parts 21a and 21b and insertion stoppers 22a and 22b are provided to a pair of earplug bodies 21 formed of a silicon rubber, respectively, holes 23a and 23b are provided on the insertion stoppers 22a and 22b, respectively, the pair of earplug bodies 21 is connected by connecting the holes 23a and 23b with a rubber cord 24, and small ring parts 25a and 25b, middle ring parts 26a and 26b, and large ring parts 27a and 27b are provided to the insertion parts 21a and 21b, respectively as shown in FIG. 11 (for example, Japanese Utility Model Laid-Open Publication No. 3-36624 (P. 1 FIG. 1)).

According to the above earplug, since the pair of earplug bodies 21 is connected with the rubber cord 24, a user will not drop only one earplug body and lose it. In addition, since the small ring parts 25a and 25b, the middle ring parts 26a and 26b, and the large ring parts 27a and 27b are provided to the insertion parts 21a and 21b, respectively, similar to the above-mentioned earplug, the earplug can be appropriate for any ear hole regardless of a diameter size of the ear hole of a user.

However, according to the conventional earplug shown in FIGS. 9 and 10, since the shaft 11 has a linear shape, when it is inserted, a rear end of the shaft 11 largely protrudes from the ear hole of a user. Furthermore, according to the earplug shown in FIG. 11 also, since the insertion parts 21a and 21b and the insertion stoppers 22a and 22b are linearly arranged, respectively, when the earplug is inserted, the insertion stoppers 22a and 22b largely protrude from the ear hole of a user.

Therefore, when these earplugs are used to prevent the water, rain, wind and the like from entering into the ear hole during sports activities, although they can prevent the water, rain, wind and the like from entering, these earplugs have problems that resistance force of the water, rain, wind and the like is applied to the rear end of the shaft 11 or the insertion stoppers 22a and 22b which largely protrude from the ear hole, and therefore a time loss is caused by the resistance force in sports competing for speed, or the earplugs are likely to be dropped off the ear because of reduction in its holding power.

In addition, when these earplugs are used to protect a sense of hearing in a noisy place, although external sound can be shut off by the earplugs, these earplugs have a problem that they cannot protect the sense of hearing satisfactorily since vibration sound generated when something touches or hits the rear end of the shaft 11 or the insertion stoppers 22a and 22b which largely protrude from the ear hole is directly transferred to the ear hole of the user as noisy sound in some cases.

Meanwhile, when an earplug is used during sports activities, in some cases it is used together with a headgear such as goggles or glasses, that is, swimming goggles for swimming, ski goggles or sunglasses for skiing, auto goggles for auto racing and the like, depending on to the kind of the sport. Furthermore, when an earplug is used in the noisy place, in some cases it is used together with a headgear such as a helmet, safety goggles, a dust proofing mask in a factory or a construction site, for example.

In those cases, if the earplug can be mounted on the headgear, even when the earplug accidentally comes off the ear hole, a user will not drop the earplug and lose it. In addition, since the earplug can be carried or kept together with the headgear, the earplug will not be left behind.

However, according to the conventional earplug shown in FIGS. 9 and 10, it is difficult to mount the earplug on the headgear. It is also difficult for the conventional earplug shown in FIG. 11 to connect the rubber cord 24 to the above-mentioned headgear.

### SUMMARY OF THE INVENTION

Therefore, the present invention was made to solve the above conventional problems and it is an object of the present invention to provide an earplug which can reduce resistance force of the water, rain, wind and the like, and reduce a time loss due to the resistance force in sports competing for speed and which is not dropped due to reduction in its holding power in case that the earplug is used to prevent the water, rain, wind and the like from entering into an ear hole during sports activities, and which can reduce vibration sound generated when something touches or hits the earplug, prevent the vibration sound from being transferred to the ear hole of a user and protect a sense of hearing satisfactorily in case that the earplug is used to protect the sense of hearing in a noisy place, and furthermore to provide a headgear with the earplug to prevent the earplug from being dropped and lost or left behind.

Thus, according to an earplug in the present invention, an earplug body 1 is connected to an arm 2 at an angle of 90±45°.

Furthermore, according to the earplug in the present invention, the arm 2 to which the earplug body 1 is connected at the angle of 90±45° is connected to one end of a cord 3.

In addition, according to an earplug in the present invention, arms 2 and 2 to which earplug bodies 1 and 1 are connected at an angle of 90±45° are connected to both ends of cord 3 respectively.

Still furthermore, according to the earplug in the present invention, the earplug body 1 and the arm 2, and the arm 2 and the cord 3 are integrally molded to be connected.

Still furthermore, according to the earplug in the present invention, hardness of the earplug body 1 has a value of 10 to 40 in a Shore A durometer hardness.

Still furthermore, according to the earplug in the present invention, a connecting part 4 is provided at the other end of the cord 3.

Still furthermore, according to the earplug in the present invention, almost the center part of the cord 3 is flattened and its width is enlarged.

Still furthermore, according to the earplug in the present invention, a connecting part 4 is provided in almost the center part of the cord 3.

Still furthermore, according to the earplug in the present invention, marks 5 and 5 are provided on appropriate positions of the cord 3 which are away from the arms 2 and 2, and connecting parts 4 are provided at the marks 5 and 5 on the side of the arms 2 and 2.

In addition, according to a headgear with an earplug in the present invention, the cord 3 of the earplug is connected thereto.

Furthermore, according to the headgear with the earplug in the present invention, the cord 3 of the earplug is connected to a band B or a temple of the headgear.

Furthermore, according to the headgear with the earplug in the present invention, when the headgear is swimming goggles G, the cord 3 of the earplug is connected to an eyecup C of the swimming goggles G.

### BRIEF DESCRIPTION OF THE DRAWINGS

FIG. 1 is a perspective view showing an earplug according to an embodiment of the present invention;

FIG. 2 is a perspective view showing an earplug according to another embodiment of the present invention;

FIG. 3 is a perspective view showing an earplug according to still another embodiment of the present invention;

FIG. 4 is an explanatory view showing a usage state of the earplug according to the present invention;

FIG. 5 is an explanatory view showing a usage state of the earplug and a headgear with the earplug according to the present invention;

FIG. 6 is an explanatory view showing a usage state of the earplug and a headgear with the earplug according to the present invention;

FIG. 7 is an explanatory view showing a usage state of the earplug according to the present invention;

FIG. 8 is an explanatory view showing a usage state of the earplug and a headgear with the earplug according to the present invention;

FIG. 9 is a side view showing an example of a conventional earplug;

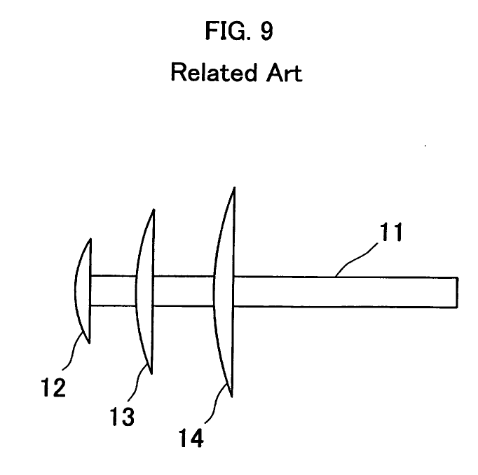

FIG. 10 is a sectional view showing the conventional earplug shown in FIG. 9; and

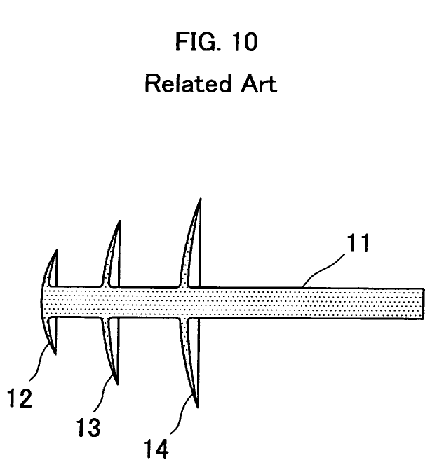

FIG. 11 is a perspective view showing an earplug according to another example of the conventional earplug.

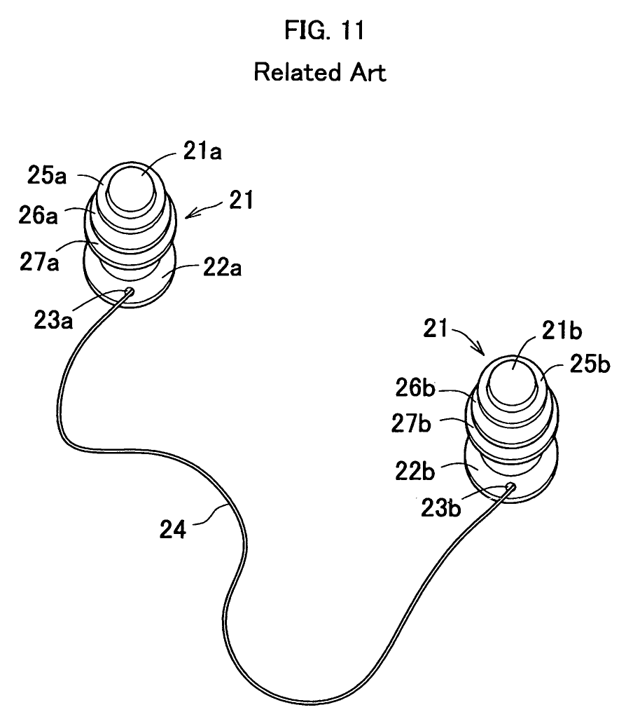

### DESCRIPTION OF THE PREFERRED EMBODIMENTS

The best mode for implementing an earplug and a headgear with the earplug according to the present invention will be described with reference to the drawings hereinafter.

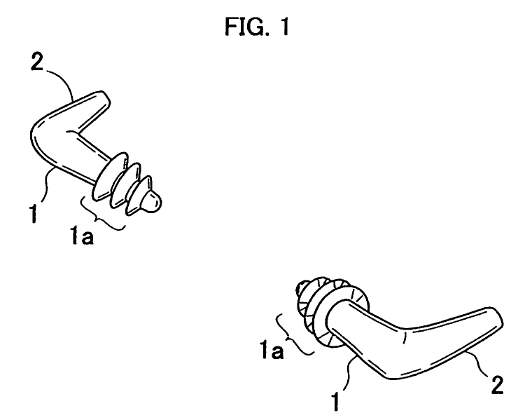

FIG. 1 shows an earplug according to an embodiment of the present invention, in which an earplug body 1 is connected to an arm 2 at a certain angle.

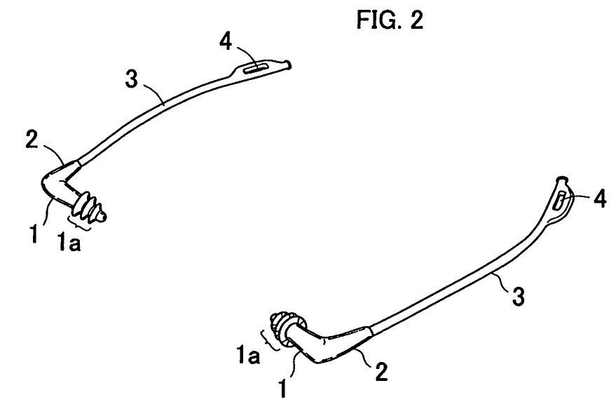

FIG. 2 shows an earplug according to another embodiment of the present invention, in which an arm 2 connected to an earplug body 1 at a certain angle is connected to one end of a cord 3.

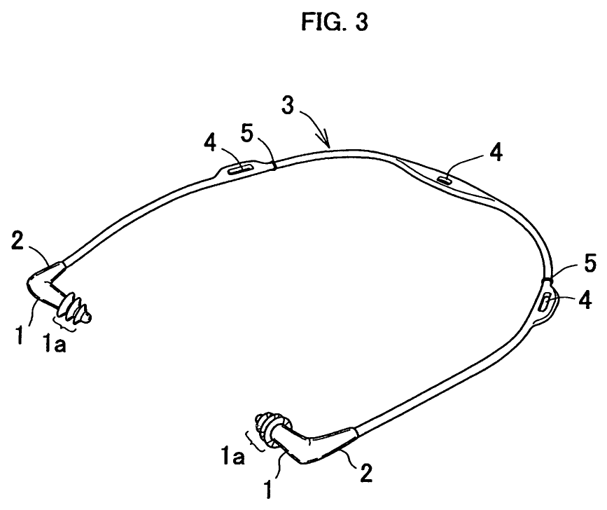

FIG. 3 shows an earplug according to still another embodiment of the present invention, in which arms 2 and 2 connected to earplug bodies 1 and 1 at a certain angle respectively are connected to both ends of cords 3.

The earplug body 1 has sound insulating performance or waterproofing performance. Although the illustrated one is a flange type in which three flanges 1a are provided to be inserted into the ear hole, the present invention is not limited to the illustrated one and the earplug body may be a plug type to be inserted into the ear hole without the flange 1a. The earplug body 1 is formed of a flexible material such as a flexible vulcanized rubber, a silicon rubber, a thermoplastic elastomer of polyolefin, polyvinyl chloride, polyurethane, polyester, polyamide, polystyrene and the like for the flange-type earplug body 1, and such as an olefin resin of self-expandable polyethylene or polypropylene for the plug-type earplug body 1. In addition, since the plug-type earplug is usually formed of the self-expandable resin, when it is integrally molded with the arm 2, the arm 2 sometimes lacks the strength, and in this case, the flange-type earplug is preferable.

The arm 2 is usually thinner than the earplug body 1, and its sectional configuration is a circle or an ellipse and it is formed of a flexible material like the earplug body 1 and may be formed of the same material as that of the earplug body 1.

The cord 3 is usually thinner than the arm 2, and its sectional configuration is a circle or an ellipse and it is formed of a flexible material like the arm 2 and may be formed of the same material as that of the arm 2.

Hardness of the earplug body 1 and the arm 2 preferably has a value of 10 to 40 in a Shore A durometer hardness. More preferably, the value is 15 to 30. When the hardness is less than 10, they are too soft and the earplug body 1 is less elastic and strength of the arm 2 is not sufficient. Meanwhile, when it is more than 40, they are so hard that it is difficult to insert the earplug body 1 into the ear hole.

The earplug body 1 and the arm 2 are preferably connected to each other at a certain angle such as 90±45°, and more preferably 90±20′. Thus, since the ear plug is inserted into the ear hole with the arm 2 in a bent state, it does not largely protrude from the ear hole of a user, so that strong resistance force is not applied thereto. In addition, since the arm 2 does not flap, the user does not feel annoying about the flapping and holding power is not reduced due to the flapping. Furthermore, since the arm 2 in the bent state comes in contact with an outer part of the ear hole of the user, vibration sound generated when something touches or hits the arm 2 is absorbed in the outer part of the ear hole and reduced so that it is not likely to be transferred to the ear hole of the user. However, in case that this angle is less than 45°, the arm 2 hits the outer part of the ear hole strongly when the earplug is inserted, so that the earplug easily comes off the ear due to its repulsive force. Meanwhile, in case that the angle is more than 135°, since the arm 2 largely protrudes from the ear hole and strong resistance force is applied thereto, the earplug easily comes off the ear hole. In addition, in the case that the angle is more than 135°, since the arm 2 does not come into contact with the outer part of the ear hole when the earplug is inserted, vibration sound cannot be reduced.

It is preferable that the earplug body 1 and the arm 2 are integrally molded to be connected in order to keep the angle between them unchanged and prevent them from being disconnected. It is also preferable that the arm 2 and the cord 3 are integrally molded in order to prevent them from being disconnected.

As shown in FIG. 2, when the arm 2 is connected to one end of the cord 3, a connecting part 4 is provided on the other end of the cord 3. This connecting part 4 may be an insertion hole provided directly in the end of the cord 3, a ring connected to the end of the cord 3, or a clip connected to the end of the cord 3. Furthermore, the connecting part 4 may be a part which functions as a pair with a connecting part (not shown) provided in the headgear such as a buckle, a clasp or a surface adhesion tape.

Thus, the cord 3 can be attached to the headgear by a connecting part 4 provided to the cord 3. In this case, in order to attach it to the headgear in a preferred state, a length of the cord 3 is preferably not more than 20 cm and more preferably 10 to 15 cm. When the length of the cord 3 is shorter than 10 cm, it cannot reach the headgear or cannot be attached thereto, and when it is longer than 20 cm, it goes slack when it is mounted on the headgear and makes the user annoying in some cases.

As shown in FIG. 3, when the arms 2 and 2 are connected to both ends of the cord 3, almost the center of the cord 3 is flattened and its width is enlarged. This flattened portion can be formed such that a width is 0.5 to 3 cm, a length is 3 to 20 cm, and a thickness is 0.5 to 3 mm and the connecting part 4 is provided in almost the center of this part. Furthermore, marks 5 and 5 are provided at a position 20 cm or less, preferably 10 to 15 cm from both ends of the arms 2 and 2, and other connecting parts 4 are provided at these marks 5 and 5 on the side of the arms 2 and 2. This connecting part 4 may be an insertion hole provided directly in the cord 3, a ring connected to the cord 3, or clip connected to the cord 3.

Thus, almost the center part of the cord 3 can be hung on the back of the neck of the user or can be attached to the headgear by the connecting part 4. In this case, it is preferable that a length of the cord 3 is 35 to 75 cm and more preferably 38 to 48 cm. When the length of the cord 3 is shorter than 35 cm, the cord 3 cannot be hung on the back of the neck of the user in some cases and when it is longer than 75 cm, it goes slack when hung on the back of the neck of the user and makes the user annoying. When almost the center part of the cord 3 is hung on the back of the neck of the user, since the cord 3 is not pulled and the arms 2 and 2 do not receive the weight of the cord 3, the earplug body 1 is not pulled and does not come off the ear hole of the user. Furthermore, when almost the center part of the cord 3 is flattened and its width is enlarged, the cord can be hung on the back of the neck of the user in a stable manner and the earplug body 1 is further prevented from coming off the ear hole of the user.

The headgear according to the present invention is integrally or detachably connected to the cord 3 of the earplug.

The headgear includes glasses such as correcting glasses, sunglasses, safety glasses, swimming goggles, ski goggles, auto goggles, safety goggles, dust proofing goggles, a head protector such as a helmet or hat, or face mask such as a safety mask, a gas mask, or a dust proofing mask.

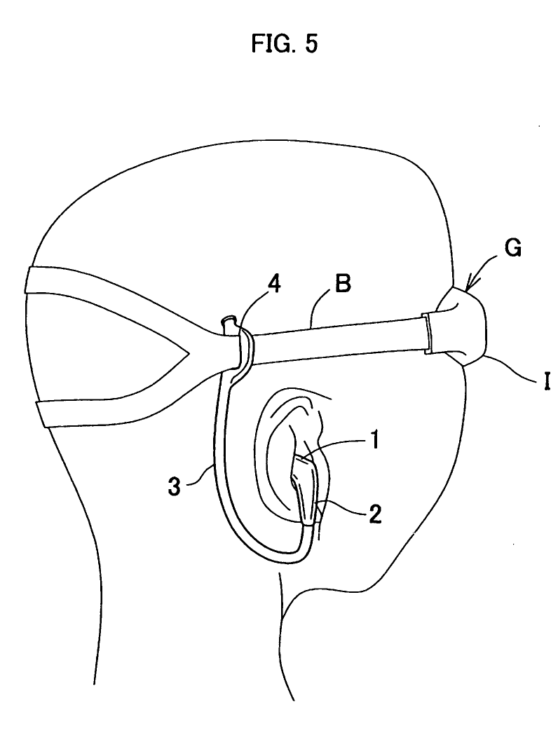

When the cord 3 of the earplug is integrally connected to the headgear, it may be directly adhered to a band B or a temple (not shown) of the headgear. In addition, when the cord 3 is detachably connected to the headgear, the band B or the temple of the headgear is inserted into the connecting part 4 provided in the cord 3, or the connecting part 4 may be hung on the band B or the temple as shown in FIGS. 5, 6 and 8. Furthermore, in case that the connecting part 4 is an insertion hole, when it is a long hole as shown in FIG. 5, the band B of the headgear is easily inserted thereto and not easily slip out. In addition, in order to connect the cord 3 to the headgear, when the headgear is swimming goggles G, the cord 3 can be connected to an eyecup C of the swimming goggles. Furthermore, the connecting part 4 can be an insertion hole provided in the cord 3 or a ring or a clip connected to the end of the cord 3. Furthermore, it may be a part functioning as a pair with a connecting part (not shown) provided on the headgear such as a buckle, a clasp or a surface adhesion tape.

Next, the earplug and the headgear with the earplug as above constituted which are in use will be explained below.

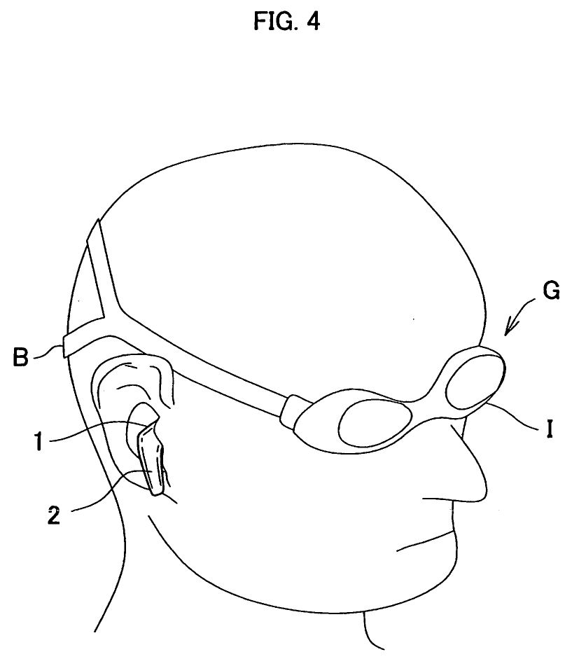

First, as shown in FIG. 4, the earplug according to the present invention shown in FIG. 1 is mounted in such a way that the earplug body 1 is inserted into the ear hole of the user with turning the arm 2 to a downward direction. Thus, since the arm 2 is in a bent state and it does not largely protrude from the ear hole of the user, resistance force of the water, rain or wind can be reduced during sports activities. In addition, since the arm 2 does not flap, the user does not feel annoying about the flapping and holding power is not reduced due to the flapping. Furthermore, since the arm 2 in the bent state comes in contact with an outer part of the ear hole of the user, vibration sound generated when something touches or hits the arm 2 is transferred to the outer part of the ear hole, so that the vibration sound transferred to the ear hole is reduced. In addition, although the earplug body 1 is inserted into the ear hole of the user in such a way to turn the arm 2 in a downward direction in FIG. 4, the earplug body 1 may be inserted in such a way to turn the arm 2 in a sideways or upward direction according to need.

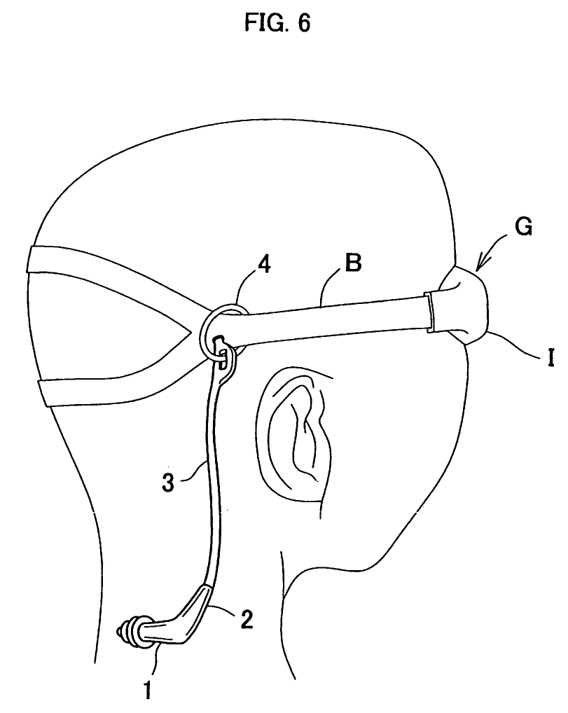

In regard to the earplug according to the present invention shown in FIG. 2, when the earplug body 1 is inserted into the ear hole of the user, in case that the connecting part 4 is the insertion hole provided in the end of the cord 3 as shown in FIG. 5, it is mounted on the swimming goggles G by inserting the band B of the swimming goggles G into the insertion hole, and in case that the connecting part 4 is the ring connected to the end of the cord 3 as shown in FIG. 6, it is mounted on the swimming goggles G by inserting the band B of the swimming goggles G into the ring. Thus, even when the earplug mounted on the ear hole as shown in FIG. 5 is removed from the ear hole as shown in FIG. 6, since the earplug is still connected to the band B of the swimming goggles G, the earplug does not fall and therefore the user will not lose the earplug. Furthermore, since the earplug can be carried or kept together with the swimming goggles G, the earplug is not left behind. In addition, the earplug of the present invention shown in FIG. 2 can be used in the same manner as the earplug of the present invention shown in FIG. 1 by removing the cord 3 from the arm 2 to have the same constitution as that of the ear plug shown in FIG. 1.

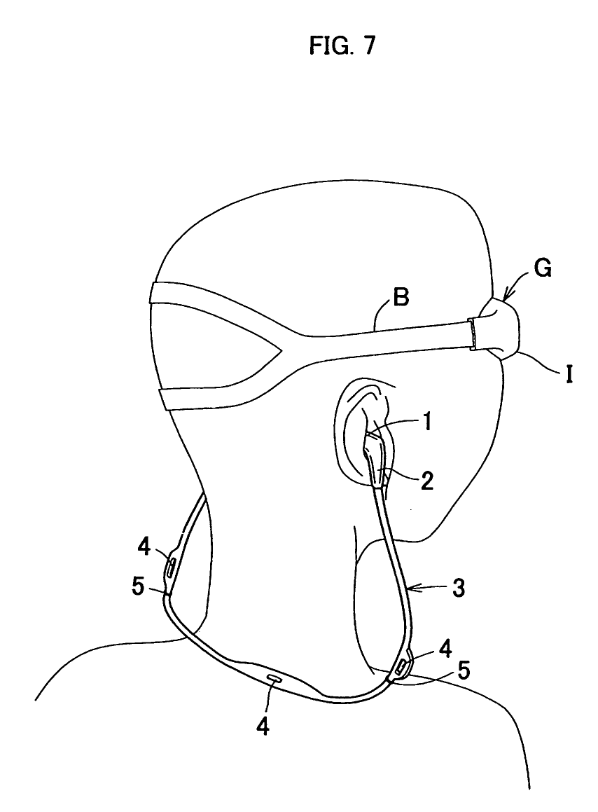

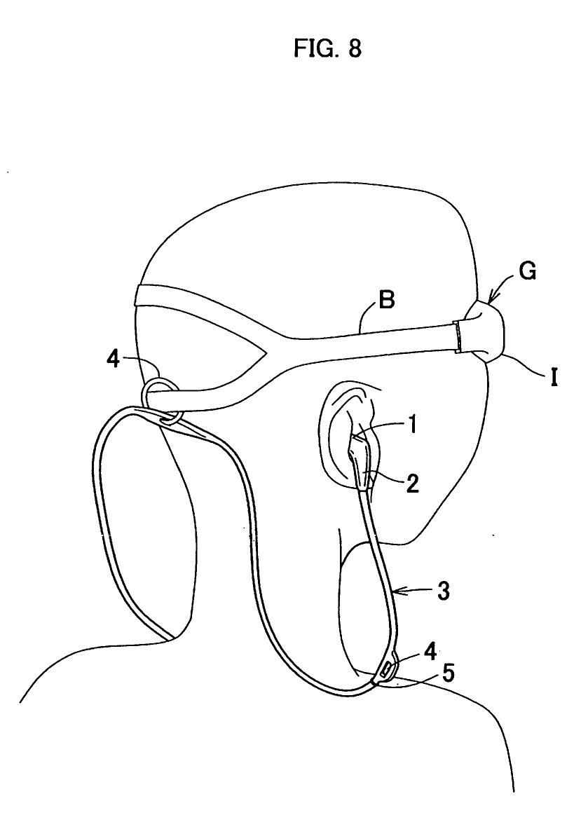

Furthermore, in case of the earplug according to the present invention shown in FIG. 3, when the earplug body 1 is inserted into the ear hole of the user, almost the center part of the cord 3 is hung on the back of the neck of the user as shown in FIG. 7, or it is mounted on the band B of the swimming goggles G, for example by the connecting part 4. Thus, in the case shown in FIG. 7, since the cord 3 is not pulled and the arms 2 and 2 do not receive the weight of the cord 3, the earplug body 1 is not pulled and it does not come off the ear hole of the user. When almost the center part of the cord 3 is flattened and its width is enlarged, since it can be hung on the back of the neck of the user stably, it is further prevented from coming off the ear hole of the user. In the case shown in FIG. 8, similar to the case in FIG. 6, even when the earplug is removed, since the earplug is still mounted on the band B of the swimming goggles G, the earplug does not fall and therefore the user will not lose the ear plug. In addition, the earplug of the present invention shown in FIG. 3 can be used in the same manner as the earplug of the present invention shown in FIG. 2 by cutting the cord 3 at the marks 5 and 5 to have the same constitution as that of the ear plug shown in FIG. 1. Furthermore, it is obvious that the earplug of the present invention shown in FIG. 3 can be used in the same manner as the earplug of the present invention shown in FIG. 1 by removing the cord 3 from the arm 2 to have the same constitution as that of the ear plug shown in FIG. 1.

The headgear with the earplug according to the present invention can be also used in the same manner as the earplug according to the present invention as shown in FIGS. 5, 6 and 8.

According to the earplug in the present invention, when it is used to prevent the water, rain, wind and the like from entering the ear hole during sports activities, resistance force of the water, rain, wind and the like is reduced. As a result, a time loss due to the resistance force can be reduced in sports competing for speed and it does not come off the ear hole because of reduction in its holding power. Furthermore, when it is used to protect a sense of hearing in a noisy place, since the vibration sound generated when something touches or hits the earplug can be reduced and it is not likely to be transferred to the ear hole of the user, so that the sense of hearing can be satisfactorily protected.

Furthermore, according to the headgear with the earplug in the present invention, the headgear is very convenient for a user since he/she will not drop and lose the earplug, or leave the earplug behind.

What is claimed is:

1. An earplug comprising:
an earplug body (1); and
an arm (2); wherein
the earplug body (1) is connected to the arm (2) at an angle of 90±45°.

2. The earplug according to claim 1 further comprising a cord (3) wherein the arm (2) is connected to one end of the cord (3).

3. An earplug comprising:
earplug bodies (1, 1);
arms (2, 2); and
a cord (3); wherein
the arms (2, 2) to which the earplug bodies (1, 1) are connected at an angle of 90±45° are connected to both ends of the cord (3).

4. The earplug according to claim 1, wherein the earplug body (1) and the arm (2) are integrally molded to be connected.

5. The earplug according to any one of claims 2 and 3, wherein the earplug body (1) and the arm (2), and the arm (2) and the cord (3) are integrally molded to be connected.

6. The earplug according to any one of claims 1 to 3, wherein hardness of the earplug body (1) has a value of 10 to 40 in a Shore A durometer hardness.

7. The earplug according to claim 2, wherein a connecting part (4) is provided at an other end of the cord (3).

8. The earplug according to claim 3, wherein almost center part of the cord (3) is flattened and its width is enlarged.

9. The earplug according to any one of claims 3 and 8, wherein a connecting part (4) is provided in almost center part of the cord (3).

10. The earplug according to any one of claims 3 and 8, wherein marks (5, 5) are provided on the cord (3) at appropriate positions from the arms (2, 2) of both ends and connecting parts (4) are provided at the marks (5, 5) on the side of the arms (2, 2).

11. The earplug according to claim 9, wherein marks (5, 5) are provided on the cord (3) at appropriate positions from the arms (2, 2) of both ends and connecting parts (4) are provided at the marks (5, 5) on the side of the arms (2, 2).

12. A headgear with an earplug, wherein the cord (3) of the earplug as set forth in any of claims 2 to 4 is connected thereto.

13. The headgear with the earplug according to claim 12, wherein the cord (3) of the earplug is connected to any one of a band (B) and a temple of the headgear.

14. The headgear with the earplug according to claim 12, wherein the headgear is swimming goggles (G) and the cord (3) of the earplug is connected to an eyecup (C) of the swimming goggles (G).
